(19) United States
(12) Patent Application Publication
(10) Pub. No.: US 2011/0015472 A1
(43) Pub. Date: Jan. 20, 2011

(54) SPHINCTER EXERCISING SYSTEM

(76) Inventor: Sinhyeong Lee, Busan (KR)

Correspondence Address: PEARNE & GORDON LLP, 1801 EAST 9TH STREET, SUITE 1200, CLEVELAND, OH, 44114-3108, US

(21) Appl. No.: 12/514,862

(22) PCT Filed: Jan. 9, 2008
(86) PCT No.: PCT/KR08/00127

§ 371 Date: May 14, 2009

(30) Foreign Application Priority Data
Nov. 13, 2007 (KR) 10-2007-0115321

(51) Int. Cl. A61F 2/00

(52) U.S. Cl. 600/29

(57) ABSTRACT

A sphincter exercising system includes a central detection unit for detecting an internal pressure of an air tube due to a constriction of a sphincter; a peripheral detection unit for detecting a pressure of a vicinity of the central detection unit; a control unit for generating an input signal for an external user terminal based on the pressures detected by the central detection unit and the peripheral detection unit; a first communication unit connected to the control unit, for outputting the input signal generated by the control unit; a second communication unit connected to the external user terminal, for receiving the input signal outputted from the first communication unit and inputting the received input signal to the external user terminal; and a power supply unit for supplying a power to the sphincter exercising system.

### TECHNICAL FIELD

The present disclosure relates to a sphincter exercising system; and, more particularly, to a sphincter exercising system that converts an air pressure variation and a movement of user's hips due to a constriction/relaxation of a sphincter into electrical signals and outputs them to an external electrical device as input signals therefor.

### BACKGROUND ART

In general, a sphincter is a muscle that surrounds a tubular organ such as an anus, a urethra, or the like and controls a discharge of excrements. The muscular strength of the sphincter degrades as a person gets older, thus causing incontinence of urine or feces. To cure or prevent such a disease, various sphincter-strengthening exercise methods and apparatuses have been proposed.

However, most of conventional sphincter exercising apparatuses are of a type that provides a stimulus to a sphincter by using a vibration generated by a driving force of a motor or the like, and with these apparatuses, a user is in a passive place in doing sphincter-strengthening exercises. Further, though it is required to motivate the user to do the exercises regularly, the conventional apparatuses do not have an auxiliary device capable of motivating the user, and it just provide simple and repetitive exercising movements.

If the user is in the passive place in doing the exercises, they may feel bored readily, failing to achieve a satisfactory result through the sphincter-strengthening exercises which are supposed to be effective only when they are repeated continually.

Moreover, some of the conventional sphincter exercising apparatuses are of a type that is inserted into the human body, thus inflicting pain on the user. This type of apparatus also has a disadvantage in that it cannot be used in an open place.

### DISCLOSURE OF THE INVENTION

### Problems to be Solved by the Invention

In view of the foregoing, the present disclosure provides a sphincter exercising system that converts an air pressure variation and a movement of user's hips due to a constriction/relaxation of a sphincter into electrical signals and outputs them to an external electrical device as input signals therefor.

### Means for Solving the Problems

In accordance with one aspect of the present invention, there is provided a sphincter exercising system including a central detection unit for detecting an internal pressure of an air tube due to a constriction of a sphincter; a peripheral detection unit for detecting a pressure of a vicinity of the central detection unit; a control unit for generating an input signal for an external user terminal based on the pressures detected by the central detection unit and the peripheral detection unit; a first communication unit connected to the control unit, for outputting the input signal generated by the control unit; a second communication unit connected to the external user terminal, for receiving the input signal outputted from the first communication unit and inputting the received input signal to the external user terminal; and a power supply unit for supplying a power to the sphincter exercising system.

In accordance with another aspect of the present invention, there is provided a sphincter exercising apparatus including a main body for supporting user's hips; a central detection unit disposed in a central portion of the main body to be brought into contact with the user's sphincter, for detecting a pressure thereat; a peripheral detection unit disposed around the central detection unit to be brought into contact with a vicinity of the user's sphincter, for detecting a pressure thereat; a control unit for generating an input signal for an external user terminal based on the pressures detected by the central detection unit and the peripheral detection unit; a communication unit connected to the control unit, for outputting an electric signal generated by the control unit to the external user terminal; and a power supply unit for supplying a power to the sphincter exercising apparatus.

In accordance with still another aspect of the present invention, there is provided a method for controlling a sphincter exercising system, including the steps of: (a) balancing user's left and right hips; (b) adhering an air tube to the user's hips: (c) detecting an internal pressure of the air tube and a pressure of a vicinity of the air tube; and (d) generating an input signal for an external user terminal based on the detected pressures.

### BRIEF DESCRIPTION OF THE DRAWINGS

The disclosure may best be understood by reference to the following description taken in conjunction with the following figures:

FIG. 1 is a block diagram schematically showing a sphincter exercising system in accordance with an embodiment of the present invention;

FIG. 2 sets forth a diagram illustrating a sphincter exercising apparatus which realizes the sphincter exercising system of FIG. 1 in accordance with the embodiment of the present invention;

FIG. 3 provides a cross sectional view taken along a line A-A of FIG. 2, which shows major components of a central detection unit of the sphincter exercising apparatus in accordance with the embodiment of the present invention;

FIG. 4 presents a diagram for schematically describing an operation of the central detection unit of FIG. 3;

FIG. 5 offers a diagram showing a peripheral detection unit of the sphincter exercising apparatus in accordance with the embodiment of the present invention;

FIG. 6 depicts a cross sectional view taken along a line B-B of FIG. 2, which shows major components of the central detection unit of the sphincter exercising apparatus in accordance with the embodiment of the present invention;

FIG. 7 is a flowchart for describing a control method of the sphincter exercising system in accordance with the embodiment of the present invention;

FIG. 8 provides a detailed flowchart for describing a method of balancing user's left and right hips; and

FIG. 9 sets forth a detailed flowchart for describing a method of firmly adhering an air tube to the user's hips.

### BEST MODE FOR CARRYING OUT THE INVENTION

Hereinafter, embodiments of the present invention will be described in detail with reference to the accompanying drawings so that the present invention may be readily implemented by those skilled in the art. However, it is to be noted that the present invention is not limited to the embodiments but can be realized in various other ways. In the drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element. Further, the term “comprises or includes” and/or “comprising or including” used in the document means that one or more other components, steps, operation and/or existence or addition of elements are not excluded in addition to the described components, steps, operation and/or elements.

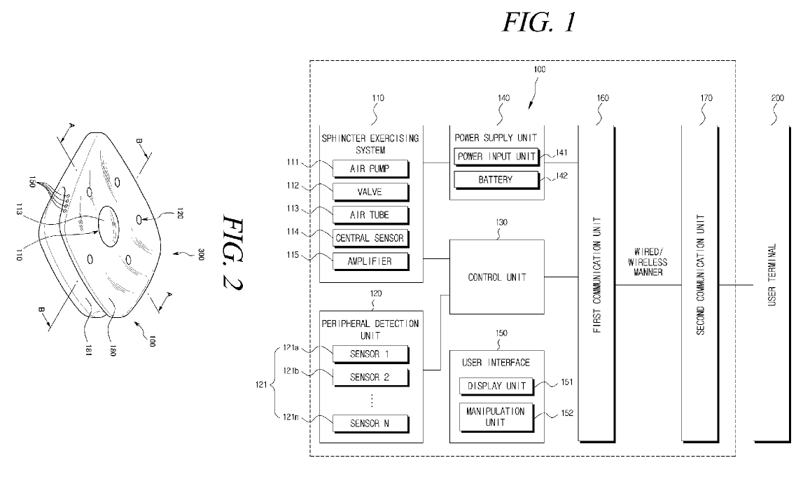

FIG. 1 is a block diagram schematically illustrating a sphincter exercising system in accordance with an embodiment of the present invention.

The sphincter exercising system 100 in accordance with the embodiment of the present invention includes a central detection unit 110, a peripheral detection unit 120, a control unit 130, a power supply unit 140, a user interface 150, a first communication unit 160 and a second communication unit 170.

The central detection unit 110 detects an internal pressure of an air tube which varies due to a constriction of a sphincter. The central detection unit 110 includes an air pump 111 for introducing air; a valve 112 connected at one end to the air pump 111, for blocking or permitting an air flow between the air pump 111 and an air tube 113 to be described next; the air tube 113 connected to the other end of the valve 112, for containing therein the air introduced by the air pump 111; a central sensor 114 connected to the air tube 113, for detecting the internal pressure of the air tube 113; and an amplifier 115 for amplifying a pressure signal of the central sensor 114.

When driven, the air pump 111 supplies air into the air tube 113, whereby the air tube 113 is expanded by the supplied air and adhered to a user's sphincter. The valve 112 is provided between the air pump 111 and the air tube 113 to serve to block or permit an air flow between the air pump 111 and the air tube 113. When the air is supplied from the air pump 111 to the air tube 113, the valve 112 is opened, and if the valve 112 is closed in the state the air tube 113 is expanded by the supplied air, the air tube 113 maintains the expanded state.

The central sensor 114 detects the internal pressure of the air tube 113, and a thus-detected pressure signal may be selectively amplified by the amplifier 115 for the accurate pressure detection.

The peripheral detection unit 120 includes a peripheral sensor set 121 having a plurality of sensors 121a to 121n. The sensors 121a to 121n are disposed around the central detection unit 110 and detect pressures around the sphincter.

A user interface 150 includes a display unit 151 for showing an operational status of the sphincter exercising system 100; and a manipulation unit 152 for receiving a user operation signal for controlling a power on/off, setting an exercise type, and so forth. The display unit 151 is a planar display such as a liquid crystal display (LCD), an organic light emitting diode (OLED), and the like. The manipulation unit 152 may be implemented by one or more buttons.

The control unit 130 controls the air pump 111 and the valve 112 based on pressure signals outputted from the central sensor 114 of the central detection unit 110 and the peripheral sensor sets 121 of the peripheral detection unit 120 and an operation signal inputted from the manipulation unit 152.

Further, the control unit 130 generates an input signal for a user terminal 200 based on the inputted pressure signals and then outputs the thus-generated input signal to the user terminal 200.

The first communication unit 160 and the second communication unit 170 are connected to the control unit 130 and the user terminal 200, respectively, thereby outputting and inputting signals between the control unit 130 and the user terminal 200. The first and the second communication unit 160 and 170 can be connected to each other in a wired or wireless manner. For example, when they are wire-connected, they may be connected by a USB (Universal Serial Bus) communication method. In the event that they are connected wirelessly, they may be connected by RF (Radio Frequency) communication, bluetooth communication, infrared data communication, wireless LAN (Local Area Network) communication, or the like, though the connection method is not limited to the aforementioned examples.

The power supply unit 140 serves to supply a power to the sphincter exercising system 100, and it includes a power input unit 141 to which a power is inputted from an external power supply, a battery 142, and so forth. The battery 142 may be disposable or rechargeable. In case the battery 142 is rechargeable, it is charged as the external power supply is connected to the power input unit 141, and even when the connection with the external power supply is cut, the battery 142 still supplies the power to the sphincter exercising system 100.

The user terminal 200 receives an electric signal outputted from the control unit 130. The received electric signal is used as an input signal for the user terminal 200. For example, the input signal may include a click signal and a direction signal. The control unit 130 converts the pressure signal from the central detection unit 110 into a click signal and the pressure signal from the peripheral detection unit 120 into a direction signal, and then outputs these signals as input signals for the user terminal 200.

Further, an input signal inputted from the user terminal 200 is transmitted to the control unit 130 of the sphincter exercising system 100 via the second and the first communication unit 170 and 160. The control unit 130 may control the sphincter exercising system 100 based on the input signal transmitted from the user terminal 200.

FIG. 2 illustrates a sphincter exercising apparatus which realizes the sphincter exercising system shown in FIG. 2 in accordance with the embodiment of the present invention.

The sphincter exercising apparatus 300 illustrated in FIG. 2 includes a main body 180, the central detection unit 110, the peripheral detection unit 120, and the user interface 150.

The main body 180 supports user's hips, and disposed in a central portion of the main body 180 is the central detection unit 110. The air tube 113 of the central detection unit 110 is configured to protrude from a top surface of the main body 180.

Disposed around the central detection unit 110 is the peripheral detection unit 120. The sensors of the peripheral detection unit 120 are located to the main body 180 along the circumference of the air tube 113 by being distanced apart from the air tube 113.

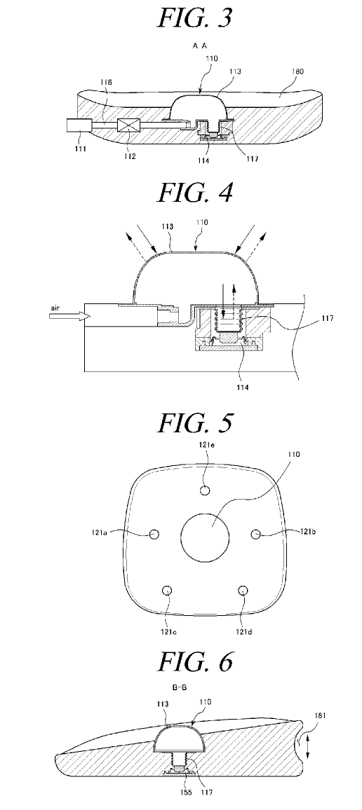

Referring to FIGS. 3 to 5, a configuration of the sphincter exercising apparatus shown in FIG. 2 will be described in detail.

FIG. 3 is a cross sectional view taken along a line A-A of FIG. 2, which shows a major configuration of the central detection unit of the sphincter exercising apparatus in accordance with the embodiment of the present invention.

As shown in FIG. 3, the air pump 111, the air tube 113 and the valve 112 are disposed in the central portion of the main body 180 of the sphincter exercising apparatus 300, and they are all connected by a first connecting member 116.

Operations of the air pump 111 and the valve 112 are controlled by the control unit (not shown). If the air pump 111 is operated in the state the valve 112 is opened, the air pump 111 introduces air into the air tube 113 through the first connecting member 116. If the valve 112 is closed, the supply of the air from the air pump 111 into the air tube 113 is stopped.

The valve 112 may be a solenoid valve, without being limited thereto. That is, any type of valve can be used as long as it is capable of blocking or permitting the air flow between the air pump 111 and the air tube 113. Moreover, the air tube 113 is made of an expansible and contractible material so that it may be expanded and contracted by the supplied air.

The central sensor 114 connected to a lower end of the air tube 113 detects an internal pressure of the air tube 113. The air tube 113 and the central sensor 114 are connected by a second connecting member 117, wherein the inner surface of the passageway of the second connecting member 117 is formed in a spiral shape to provide elasticity against a force applied in a vertical direction. Accordingly, the sphincter exercising apparatus 300 can be prevented from being damaged by an impact in the vertical direction.

The internal pressure of the air tube 113 detected by the central sensor 114 is transmitted to the control unit (not shown) to be used as a basic data for controlling the sphincter exercising apparatus 300.

FIG. 4 provides a diagram for schematically describing an operation of the central detection unit shown in FIG. 3.

As illustrated in FIG. 4, if the air pump 111 is operated in the state the valve 112 is opened, the air tube 113 is expanded due to an inflow of air, and the expanded air tube 113 adheres to the vicinity of a user's sphincter.

The internal pressure of the air tube 113 is detected by the central sensor 114, and a thus-detected pressure signal is inputted to the control unit 130. Here, for the accurate pressure detection, the amplifier 115 may be disposed between the central sensor 114 and the control unit 130. If the internal pressure of the air tube 113 reaches a preset value or greater by operating the air pump 111 in the state the valve 112 is opened, the control unit 130 closes the valve 112 to stop the supply of the air into the air tube 113 and then stops the operation of the air pump 111 as well.

Accordingly, the air tube 113 configured to protrude from the central top surface of the main body 180 can be adhered to the vicinity of the user's sphincter, while maintaining a constant pressure level therein.

If the air tube 113 is compressed due to a constriction of the sphincter while the internal pressure of the air tube 113 is regulated at the constant level, the internal pressure of the air tube 113 increases, and this increased pressure is detected by the central sensor 114.

If a thus-detected pressure signal is inputted from the central detection unit 110 to the control unit 130, the control unit 130 determines whether the constriction of the sphincter has taken place, and outputs an electric signal corresponding to the determination result to an external user terminal (not shown) via the first communication unit 160.

In case the external user terminal is a computer, for example, the electric signal corresponding to the constriction of the sphincter may be outputted as a click signal of a mouse or an enter key signal of a keyboard.

As described above, by detecting the internal pressure of the air tube 113 while keeping the air tube 113 adhered to the user's sphincter by way of expanding the air tube 113 with the air pump 111 and, then, by determining a constriction of the sphincter based on pressure signals, the sphincter exercising apparatus 300 in accordance with the embodiment of the present invention can detect sphincter movements more precisely.

FIG. 5 illustrates the peripheral detection unit of the sphincter exercising apparatus in accordance with the embodiment of the present invention.

As shown in FIG. 5, the peripheral sensor set 121 of the peripheral detection unit 120 includes five sensors 121a to 121e that are disposed around the central detection unit 110.

The peripheral sensor set 121 serves to detect a state and a movement of hips around the sphincter. Pressure signals detected by the peripheral sensor set 121 can be used by the control unit 130 as a basic data of a direction signal for the user terminal 220. Further, based on the distribution of the pressure signals detected by the peripheral sensor set 121, the control unit 130 can determine whether the user is properly seated on the main body 180 of the sphincter exercising apparatus 300 or whether the user leans forward/backward.

In the embodiment, the peripheral sensor set 121 includes two compensation sensors 121a and 121b to detect the seated state of the user and three direction sensors 121c to 121e to detect a movement direction of the user's hips, three direction sensors 121c to 121e having two horizontal sensors 121c and 121d and a vertical sensor 121e. However, it is to be noted that the number of the sensors of the peripheral sensor set 121 and their installation places are not limited to those described in the embodiment.

Specifically, the compensation sensors 121a and 121b respectively located at left and right sides of the central detection unit 110 are brought into contact with left and right hips of the user, and detect pressures by the left and right hips.

Pressure signals from the compensation sensors 121a and 121b are inputted to the control unit 130. The control unit 130 compares the pressure signals inputted from the compensations sensor 121a and the compensation sensor 121b, and determines whether the user is properly seated on the sphincter exercising apparatus 300. Then, abnormality of the seated state of the user, if any, can be outputted by means of the user interface 150.

Meanwhile, two horizontal sensors 121c and 121d located at the front side of the central detection unit 110 are brought into contact with left and right thighs, respectively, and detect pressures by the left and right thighs.

Pressure signals outputted from the horizontal sensors 121c and 121d are inputted to the control unit 130. The control unit 130 detects movements of the left and right thighs of the user based on the pressure signals inputted from the horizontal sensors 121c and 121d.

The pressure signals from the horizontal sensors 121c and 121d may be converted into electric signals by the control unit 130 to be used as left and right directional signals for the user terminal 200.

Further, the vertical sensor 121e located at the rear side of the central detection unit 110 comes into contact with the upper portion of the user's hips and detects a pressure by a user's weight.

A pressure signal outputted from the vertical sensor 121e is inputted to the control unit 130. The control unit 130 determines whether the user leans forwardly or backwardly based on the inputted pressure signal.

The pressure signal detected by the vertical sensor 121e is converted into an electric signal by the control unit 130 to be used as an upward or a downward direction signal for the user terminal 200.

FIG. 6 sets forth a cross sectional view taken along a line B-B of FIG. 2, which shows a major configuration of the central detection unit of the sphincter exercising apparatus in accordance with the embodiment of the present invention.

As illustrated in FIG. 6, the top surface of the main body 180 is inclined upward at a preset angle with the bottom surface thereof. Further, a groove portion 181 is formed in the rear surface of the main body 180, and the main body 180 may be formed of an elastic material.

Since the top surface of the main body 180 is inclined at the preset angle with the bottom surface thereof, the user can be seated on the sphincter exercising apparatus 300 in a more comfortable posture. Moreover, since the groove portion 181 is provided in the rear surface of the main body 180, the top surface of the sphincter exercising apparatus 300 is allowed to make vertical movements elastically within a specific range. Therefore, an impact applied on the hips of the user through the top surface of the sphincter exercising apparatus 300 can be absorbed.

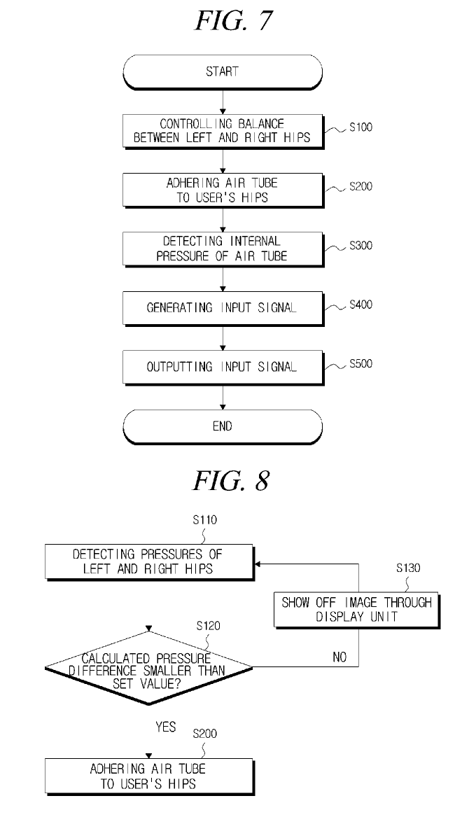

Referring to FIG. 7, there is provided a flowchart for describing a control method of the sphincter exercising system in accordance with the embodiment of the present invention.

If the user sits on the sphincter exercising apparatus and turns on the apparatus, a balance between the left and right hips of the user is controlled based on pressure signals detected from the compensation sensors of the peripheral detection unit (S100).

The step S100 of balancing the left and right hips of the user will be explained in further detail, with reference to FIG. 8. With the sphincter exercising apparatus turned on, the two compensation sensors detect pressures by the left and right hips of the user, respectively (S110).

The control unit calculates a difference between the pressure values detected by the compensation sensors, and compares the calculated pressure difference with a set value (S120).

If the pressure difference exceeds the set value, the control unit concludes that the user is seated in the unbalanced state of the left and right hips, and sets forth a sound or shows off an image through the display unit of the user interface (S130).

After completing the step S100 for balancing the left and right hips of the user, the air tube is expanded and adhered to the user's hips (S200).

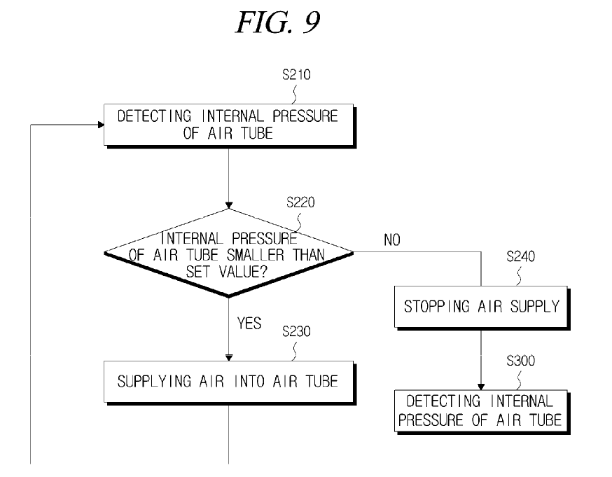

The step S200 of adhering the air tube to the user's hips will be explained more specifically with reference to FIG. 9. First, an internal pressure of the air tube is detected by the central sensor (S210), and it is determined whether the detected internal pressure of the air tube is no smaller than a set value (S220). If the internal pressure of the air tube is smaller than the set value, the control unit opens the valve and operates the air pump to thereby supply air into the air tube (S230).

Once the air is supplied by the air pump, the internal pressure of the air tube increases, and the control unit continually detects whether the internal pressure of the air tube detected by the central sensor is no smaller than the set value (S220).

If the internal pressure of the air tube is equal to or greater than the set value, the control unit concludes that the air tube is firmly adhered to the user's hips, and stops the air supply into the air tube (S240). The air supply can be stopped by stopping the air pump and closing the valve.

If the air tube is firmly adhered to a recessed portion between the user's hips while the internal pressure of the air tube is maintained constant, the central sensor of the central detection unit detects an internal pressure of the air tube, while the peripheral sensor set of the peripheral detection unit detects pressures of the vicinity of the user's hips (S300).

Thus detected pressure signals are inputted to the control unit and converted into input signals for the external user terminal (S400).

For example, in the event that a pressure value detected by the central sensor increases beyond a preset pressure range, the control unit concludes that the user has constricted a sphincter, and converts the pressure signal into an electric signal corresponding to the determination result.

Further, in the event that a pressure value detected by the left horizontal sensor increases beyond a preset pressure range, the control unit concludes that the user has pressed the left thigh portion. Meanwhile, in the event that a pressure value detected by the right horizontal sensor increases beyond a preset pressure range, the control unit concludes that the user has pressed the right thigh portion. Then, the control unit converts those pressure signals into electric signals corresponding to determination results.

Thereafter, the electric signals converted by the control unit are outputted to the external user terminal via the first communication unit which is connected with the control unit (S500). The external user terminal receives, as input signals, the electric signals outputted from the control unit via the first communication unit.

For example, assuming that the external user terminal is a computer or a game device, if an electric signal corresponding to a click of a mouse is outputted by the control unit, the same effect as the clicking of the mouse can be obtained as the external device receives this electric signal.

Meanwhile, though the above embodiment has been described for the case of transmitting the electric signal to the external device in a wireless manner, it is also possible to output it by a wire connection. Furthermore, though the step S100 of balancing the left and right hips of the user is performed only once in the above-described embodiment, it is also preferable to perform the balancing operation for every step of the process.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention.

The scope of the present invention is defined by the following claims rather than by the detailed description of the embodiment. It shall be understood that all modifications and embodiments conceived from the meaning and scope of the claims and their equivalents are included in the scope of the present invention.

### INDUSTRIAL APPLICABILITY

In accordance with the embodiment of the present invention described above, there is provided the sphincter exercising apparatus which converts an air pressure variation and a movement of user's hips due to a constriction/relaxation of a sphincter into electrical signals and outputs them as input signals for an external electric device. Accordingly, the user is allowed to do sphincter-strengthening exercises effectively without feeling a sense of boredom.

1. A sphincter exercising system comprising:
a central detection unit for detecting an internal pressure of an air tube due to a constriction of a sphincter;
a peripheral detection unit for detecting a pressure of a vicinity of the central detection unit;
a control unit for generating an input signal for an external user terminal based on the pressures detected by the central detection unit and the peripheral detection unit;
a first communication unit connected to the control unit, for outputting the input signal generated by the control unit;
a second communication unit connected to the external user terminal, for receiving the input signal outputted from the first communication unit and inputting the received input signal to the external user terminal; and
a power supply unit for supplying a power to the sphincter exercising system.

2. The sphincter exercising system of claim 1, wherein the central detection unit includes:
an air pump for introducing air;
an air tube for containing therein the air introduced by the air pump;
a valve disposed between the air pump and the air tube, for permitting or blocking an air flow therebetween; and
a central sensor connected to a lower portion of the air tube, for detecting the internal pressure of the air tube.

3. The sphincter exercising system of claim 2, wherein the central detection unit further includes an amplifier for amplifying a pressure value detected by the central sensor.

4. The sphincter exercising system of claim 1, wherein the peripheral detection unit includes a plurality of sensors.

5. The sphincter exercising system of claim 1, further comprising a user interface unit for showing an operational status of the system or receiving an operation signal from a user.

6. (canceled)

7. The sphincter exercising system of claim 1, wherein the control unit controls the air pump and the valve based on the internal pressure of the air tube detected by the central detection unit.

8. (canceled)

9. (canceled)

10. (canceled)

11. (canceled)

12. (canceled)

13. A sphincter exercising apparatus comprising:
a main body for supporting user's hips;
a central detection unit disposed in a central portion of the main body to be brought into contact with the user's sphincter, for detecting a pressure thereat;
a peripheral detection unit disposed around the central detection unit to be brought into contact with a vicinity of the user's sphincter, for detecting a pressure thereat;
a control unit for generating an input signal for an external user terminal based on the pressures detected by the central detection unit and the peripheral detection unit;
a communication unit connected to the control unit, for outputting an electric signal generated by the control unit to the external user terminal; and
a power supply unit for supplying a power to the sphincter exercising apparatus.

14. The sphincter exercising apparatus of claim 13, wherein the main body has a recessed central portion lower than an edge portion thereof.

15. The sphincter exercising apparatus of claim 13, wherein a top surface of the main body inclines upward at a preset angle with a bottom surface thereof.

16. The sphincter exercising apparatus of claim 13, wherein the main body is provided with a concave groove at a rear surface thereof.

17. The sphincter exercising apparatus of claim 13, wherein the central detection unit includes:
an air pump for introducing air;
an air tube protruded from a central top surface of the main body to be brought into contact with the user's hips;
a first connecting member connecting the air pump and the air tube;
a valve installed on the first connecting member between the air pump and the air tube;
a central sensor connected to a lower portion of the air tube, for detecting an internal pressure of the air tube; and
a second connecting member connecting the air tube and the central sensor.

18. The sphincter exercising apparatus of claim 13, wherein the central detection unit further includes an amplifier for amplifying the pressure signal detected by the central sensor.

19. The sphincter exercising apparatus of claim 13, wherein the peripheral detection unit includes a plurality of sensors.

20. The sphincter exercising apparatus of claim 19, wherein the sensors include compensation sensors disposed at a left and a right side of the central detection unit, respectively.

21. The sphincter exercising apparatus of claim 19, wherein the sensors include two horizontal sensors disposed at a front side of the central detection unit.

22. The sphincter exercising apparatus of claim 19, wherein the sensors include one vertical sensor disposed at a rear side of the central detection unit.

23. The sphincter exercising apparatus of claim 13, further comprising a user interface disposed at a side surface of the main body, for showing an operational status of the sphincter exercising apparatus or receiving an operation signal from the user.

24. (canceled)

25. (canceled)

26. (canceled)

27. (canceled)

28. (canceled)

29. (canceled)

30. A method for controlling a sphincter exercising system, comprising the steps of:
(a) balancing user's left and right hips;
(b) adhering an air tube to the user's hips:
(c) detecting an internal pressure of the air tube and a pressure of a vicinity of the air tube; and
(d) generating an input signal for an external user terminal based on the detected pressures.

31. The method of claim 30, further comprising a step of outputting the generated input signal to the external user terminal.

32. The method of claim 30, wherein the step (a) includes the steps of:
(a-1) detecting pressures of the user's left and right hips; and
(a-2) comparing a pressure difference between the left and right hips with a set value.

33. (canceled)

34. The method of claim 30, wherein the step (b) includes:
(b-1) detecting an internal pressure of the air tube; and
(b-2) controlling an air pump and an air valve based on the detected internal pressure of the air tube.

35. (canceled)

36. (canceled)

37. The method of claim 30, wherein the step (c) includes:
(c-1) detecting an internal pressure of the air tube;
(c-2) detecting a pressure of a left vicinity of the air tube;
(c-3) detecting a pressure of a right vicinity of the air tube; and
(c-4) detecting a pressure of a rear vicinity of the air tube.

38. The method of claim 37, wherein the step (d) includes a step of generating an input signal corresponding to a click of a mouse or an enter key of a keyboard when the detected internal pressure of the air tube is greater than or equal to a set value.

39. (canceled)

40. (canceled)

41. (canceled)

42. (canceled)
